(19) United States
(12) Patent Application Publication
(10) Pub. No.: US 2015/0238646 A1
(43) Pub. Date: Aug. 27, 2015

(54) METHOD FOR CLEANING, DISINFECTING AND/OR STERILISING PACKAGING MEANS AND/OR COMPONENTS IN CONTAINER TREATMENT SYSTEMS

(71) Applicant: KHS GMBH, Dortmund (DE)

(72) Inventor: Diana Wolf, Miehlen (DE)

(21) Appl. No.: 14/434,884

(22) PCT Filed: Aug. 30, 2013
(86) PCT No.: PCT/EP2013/002608

§ 371 Date: Apr. 10, 2015

(30) Foreign Application Priority Data
Oct. 12, 2012 (DE) 10 2012 109 758.5
Nov. 26, 2012 (DE) 20 2012 011 289.9

(51) Int. Cl. A61L 2/18; B65B 55/10; B08B 9/08

(52) U.S. Cl. CPC A61L 2/18; B08B 9/08; B65B 55/10

(57) ABSTRACT

A method for treating a structure includes cleaning, disinfecting, or sterilizing the structure. The structure can be a food package, or a critical region of a system component of a container treatment system. Treating the structure includes treating it with an aqueous solution of chlorine dioxide having a chlorine dioxide concentration that is between 0.4 ppm-0.8 ppm.

### RELATED APPLICATIONS

This application is the national stage under 35 USC 371 of PCT/EP2013/002608, filed Aug. 30, 2013, which claims the benefit of the Oct. 12, 2012 priority date of DE 10-2012-109-758.5 and the benefit of the Nov. 26, 2012 of DE 20-2012-011-289.9, the contents of both of which are herein incorporated by reference.

### FIELD OF DISCLOSURE

The invention relates to cleaning and disinfecting packages.

### BACKGROUND

Filling machines used for filling containers with food products are periodically cleaned and disinfected to avoid introducing microorganisms into food. This is especially important for surfaces that come into contact with the food product. It is also useful to clean, disinfect, and/or sterilize packages to be filled with the food product, seals, and/or the preforms before the packages are filled.

Products vary in their sensitivity to fouling as a result of bacterial contamination. Such microbiologically sensitive products include those that lack preservatives, those with alcohol content between 0% and 0.5% by volume, those with a carbon dioxide content of between 0 grams per liter and 5 grams per liter, and those with a pH greater than 4. These parameters can influence one another and only represent guidelines concerning what is and is not microbiologically sensitive product

For the filling of microbiologically sensitive products it is known to treat components of a system, and in particular, its critical regions, with peracetic acid. This reduces contamination of those regions by microorganisms or bacteria to an extent that significantly suppresses any negative effects on the filled packages. The package itself is also treated with peracetic acid as the treatment medium. This treatment is preferably carried out by a rinser that sprays the package for six to eight seconds with a 40° C. aqueous solution of peracetic acid dissolved at a concentration of 600-800 ppm, sprays the containers twice with sterile water for approximately half a second each time, and allows the package to drain or drip off for 3.5 to 4 seconds.

A disadvantage of the known method is that peracetic acid attacks the stainless steel of the system components. This leads to corrosion. The known method also results in long treatment times, in some cases around fourteen seconds. During this time, the treatment medium must be maintained at 40° C. This imposes energy costs. Additionally, the overall apparatus to carry out these functions is technically complex.

Additional disadvantages arise from the need to extract vapors released from the peracetic acid, and because peracetic acid breaks down to form acetic acid. Microorganisms can then use this acetic acid as a nutritive medium.

### SUMMARY

An object of the invention is to provide a method that avoids the foregoing disadvantages while also achieving a reduction in the treatment time or duration.

Surprisingly it has been shown that when an aqueous solution of chlorine dioxide having a concentration of less than 0.8 ppm in the aqueous solution of chlorine dioxide is used as the treatment medium, it is possible to execute a treatment of a package with the required quality. This treatment extends not just to the package, especially when the product that is filled into the cleaned and sterilized package has a CO₂ content of more than 4 g/L. It also extends to system components. Despite the low chlorine dioxide concentration, the treatment process has an adequate microbicidal effect on undesirable microorganisms and bacteria of all kinds, including in particular those microorganisms and bacteria that could cause microbiological deterioration in drinks with a CO₂ content of less than 5 g/L. The disadvantages of corrosion of the stainless steel in the system components and of a malodorous environment are significantly reduced by the inventive method.

The invention is based in part on the realization that the application time of the treatment medium formed by the aqueous solution of chlorine dioxide, i.e. the treatment time in the treatment steps in question, can be significantly shortened without impairing or entirely sacrificing the desired effect. Thus, for example an application time or treatment time of 1-2 seconds is fully sufficient to treat the package while an application time or treatment time of 5 to 10 minutes is fully sufficient to treat the critical regions of the system components.

In one aspect, the invention includes a method comprising treating structure by either cleaning, disinfecting, or sterilizing it. The structure can be a food package or a critical region of a system component of a container treatment system. Treating the structure includes selecting an aqueous solution of chlorine dioxide having a chlorine dioxide concentration that is between 0.4 ppm-0.8 ppm as a treatment medium, and, in at least one treatment step, treating the structure with this treatment medium.

In some practices, treating the structure with the treatment medium comprises using the treatment medium in at least a first treatment step and a second treatment step that follows the first. In the first treatment step, the treatment medium has a chlorine dioxide concentration between 0.4 ppm and 0.8 ppm. In the second treatment step, the treatment medium has a chlorine dioxide concentration between 0.2 ppm and 0.4 ppm. Some of these practices include causing the first treatment step to last between one and two seconds, and causing the second treatment step to last half a second. Others include causing the first treatment step to last between one and two seconds, and causing the second treatment step to last between one and two seconds.

Some practices feature using the treatment medium in at least a first treatment step that has a treatment time between one and two seconds. Other practices include those in which treating the structure with the treatment medium comprises causing the at least one treatment step to last between five minutes and ten minutes.

Among the practices of the invention are those in which structure to be a food package, and in particular, a container that is either unsoiled or cleaned. In these practices, a first treatment step comprises the step of treating the structure with the treatment medium. Two other treatment steps follow in sequence. The second treatment step comprises a treatment step that uses an aqueous solution of chlorine dioxide, and the third includes removing the aqueous solution of chlorine dioxide. Among practices of this method are those in which removing the aqueous solution of chlorine dioxide comprises allowing the aqueous solution to drip off the container, and those in which removing the aqueous solution of chlorine dioxide comprises rinsing off the container, either with a sterile liquid or with a gaseous medium.

Other practices of the invention include selecting the structure to be at least one of a keg and a keg fitting.

In other practices in which the structure has a keg and a keg fitting, the method includes, during treatment of the structure, draining the keg fitting to remove product residues, in a first subsequent treatment step causing the keg fitting to undergo an internal cleaning, and in a second subsequent treatment step, causing the structure to be either cleaned, disinfected, or sterilized with aqueous solution of chlorine dioxide. Causing the keg to undergo internal cleaning includes either treating the keg with a base, treating the keg with an acid, purging the keg with a base, purging the keg with an acid, flushing the keg with water, or flushing the keg with fresh water.

Other practices that include treating a keg include releasing a ring seal from a location between the keg fitting and an inner surface of the keg, thereby exposing an additional surface for treatment, and treating the additional surface with the treatment medium.

Yet other practices include selecting the structure to comprise system components of a filling machine. This method includes treating critical regions of the system components in a first treatment step that comprises treatment either an acid or a base, and treating the critical regions in a second treatment step with the aqueous solution of chlorine dioxide. Among these practices are those that include selecting the aqueous solution to have a chlorine dioxide concentration of between 0.2-0.4 ppm and those that include selecting at least one of the treatment steps to have a treatment duration of between five and ten minutes.

As used herein, “critical regions” of the system components are such regions and/or surfaces of the machines or components of a system that come into contact, during filling, with the product and/or with a treatment medium for the pre-treatment of the package before they are filled.

For the purpose of the invention, “package” refers to any packages or containers usually used in the food industry, and in particular, containers such as for example bottles, cans, as well as soft packages, for example those produced from cardboard and/or plastic film and/or metal film etc.

As used herein, “substantially” or “around” mean variations from an exact value that are insignificant for the function associated with the exact value.

As used herein, “treatment” includes cleaning, disinfection, sterilization, and any combination thereof.

As used herein, “treatment medium” includes any medium used to achieve treatment.

Further embodiments, advantages and possible applications of the invention arise out of the following description of embodiments and out of the figures. All of the described and/or pictorially represented attributes whether alone or in any desired combination are fundamentally the subject matter of the invention independently of their synopsis in the claims or a retroactive application thereof. The content of the claims is also made an integral part of the description.

### BRIEF DESCRIPTION OF THE DRAWINGS

These and other features of the invention will be apparent from the following detailed description and the accompanying figures, in which:

FIG. 1 shows a simplified flowchart depicting treatment steps for treating containers or bottles;

FIG. 2 shows a simplified flowchart depicting treatment steps for treating kegs; and

FIG. 3 shows a simplified flowchart depicting treatment steps for treating critical regions of system components of container treatment systems, in particular of cleaning machines, rinsing machines, filling machines, sealing machines, devices for sterilizing container seals, and devices for sterilizing preforms.

### DETAILED DESCRIPTION

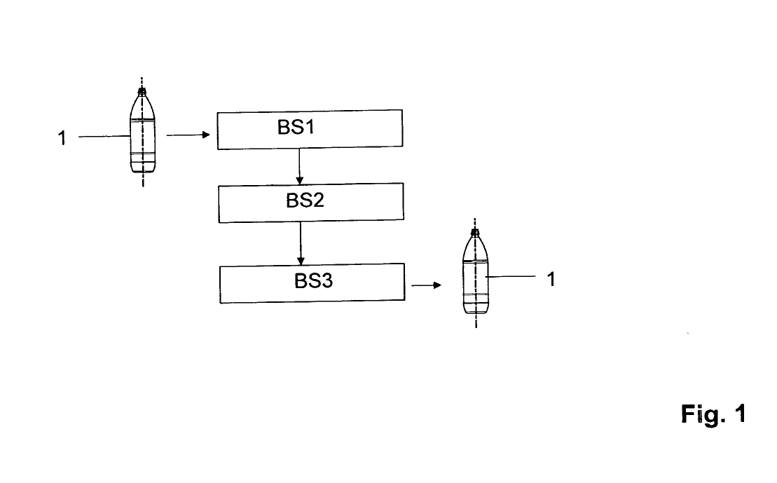

FIG. 1 shows the main treatment steps of a method for internal treatment of a container 1 that is subsequently to be filled with a product. Typically, the product is one that is highly sensitive to microbiological fouling or contamination that is harmful to the product.

According to FIG. 1, the treatment of a container 1 includes a first treatment step BS1, a second treatment step BS2, and a third treatment step BS3. These steps are typically carried out in a container-treatment machine, and in particular, in a rinsing machine.

The first treatment step BS1 includes spraying a first treatment medium into the interior of the container 1. Preferably, the first treatment medium is an aqueous solution of chlorine dioxide having a chlorine dioxide concentration of between 0.4 ppm and 0.8 ppm. The treatment time for the first treatment step BS1 is preferably only between one and two seconds inclusive.

The second treatment step BS2 chronologically follows the first treatment step BS1. The second treatment step BS2 includes spraying the interior of the container 1 with an aqueous solution of chlorine dioxide. In the second treatment step BS2, the chlorine dioxide concentration is within a range between 0.2 ppm and 0.4 ppm. In some embodiments, the treatment time associated with the second treatment step is between one and two seconds inclusive. However, in other embodiments, the treatment time is only half a second.

The third treatment step BS3 chronologically follows the second treatment step BS2. The third treatment step BS3 includes completely emptying the container 1 and allowing it to drip dry.

In the case of the method described above, it was assumed that unsoiled containers 1 are available from the beginning. An example of such a container is one that has been blow molded immediately prior to treatment. This container could be a PET container, such as a PET bottle.

The method is also adaptable for use with reusable containers by executing a cleaning step before the first treatment step BS1. The cleaning step is carried out with the usual cleaning methods and/or in a usual container-cleaning machine.

Optionally, an external cleaning step is also carried out. This external cleaning step can be carried out during the first and second treatment steps BS1, BS2 or during a further treatment step that follows the second treatment step BS2. The external cleaning step cleans and/or disinfects the container's outer surface. Preferably, external cleaning is carried out at least in such regions of the container's outer surface that present the risk of contamination of the product during container-filling and during container-sealing.

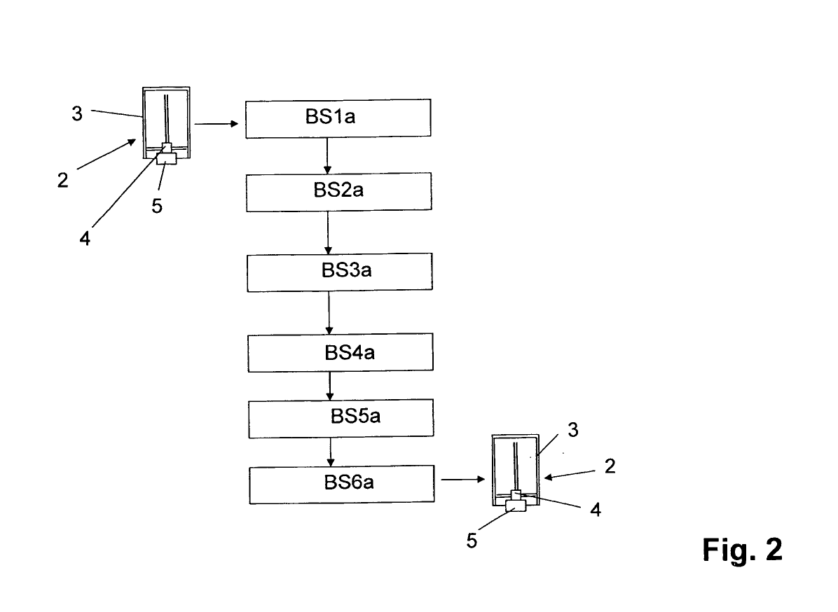

In some cases, a container 2 comprises a keg 3 having a keg-fitting 4, as shown in FIG. 2. A keg 3 comprises a large-volume container body 3 made, for example, from metal or plastic (PET). A keg-fitting 4 located on the top of the keg 3 has a valve.

FIG. 2 shows treatment steps associated with treatment of a keg's interior and/or its keg-fitting 4. The illustrated procedure begins with turning a keg 3 upside-down and setting the keg-fitting 4 thereof on a treatment head 5.

A first treatment step BS1a includes opening the keg-fitting 4 and emptying the keg 3 of any product residues through the treatment head 5.

A second treatment step BS2a includes a rinsing the keg's interior with a rinsing or cleaning fluid though the treatment head 5 and the opened keg-fitting 4. A suitable rinsing or cleaning fluid includes either a base or an acid.

A third treatment step BS3a includes rinsing the keg's interior with water through the treatment head 5 and the opened keg-fitting 4.

Fourth and fifth treatment steps BS4a, BS5a include actual treatment of the keg's interior and of all surfaces or regions of the keg-fitting 4 that come into contact with the product. This includes applying treatment medium to such surfaces or regions. A suitable treatment medium is an aqueous chlorine dioxide solution. The treated regions include all surfaces and regions of the keg-fitting 4.

In the fourth treatment step BS4a, the aqueous chlorine dioxide solution has a chlorine dioxide concentration in the range between 0.4 ppm and 0.8 ppm. The duration of the fourth treatment step BS4a is, for example, between one and two seconds.

In the fifth treatment step, BS5a, the aqueous chlorine dioxide solution has a chlorine dioxide concentration in the range between 0.2 and 0.4 ppm. The duration of the fifth treatment step BS5a can be around 1-2 seconds. However, in some preferred practices, the duration is only 0.5 seconds.

A complete draining of the treatment medium from the keg's interior is then effected in a sixth treatment step BS6a with the keg 3 disposed on the treatment head 5 and the keg-fitting 4 open.

A ring seal is usually arranged between the keg-fitting 4 and the keg's inside surface. This ring seal normally seals a local transition between the keg-fitting 4 and the keg's body.

During either the fourth treatment step B4a or the fifth treatment step B5a, the treatment head 5 acts upon the keg-fitting 4 in such a way as to lift this ring seal off the inside surface. With the ring seal thus lifted, a short pulse of treatment medium is introduced into container 2. This pulse of treatment medium flows between the ring seal and the inner surface of the keg's body and back into treatment head 5 so that surfaces exposed by having lifted the ring seal can also be treated with the treatment medium. These exposed surfaces include a surface on the inner surface of container body 3 and a surface on the ring seal itself.

Following execution of the methods shown in FIG. 1 and FIG. 2, it may be expedient to rinse and dry the interior of a container 1, 2 after having treated it. This rinsing can be carried out by rinsing with a sterile medium. Such a rinsing step removes chlorine dioxide residues. A suitable sterile medium is sterile water. After rinsing, purging the container with a sterile gaseous medium dries the container 1, 2.

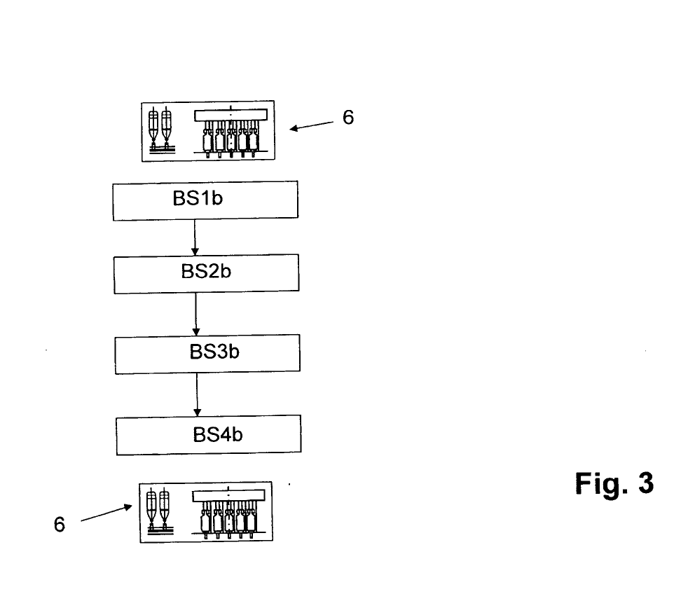

FIG. 3 shows steps associated with treatment of system components 6, such as those found in a rinsing machine or in a filling machine. In the case of systems for the filling of sensitive products in containers, it is necessary to treat the relevant system components 6 in their critical regions at certain intervals.

A first step BS1b of the process includes stopping normal production of the process system.

A second step BS2b of the process includes treating all critical regions of the system components 6 with base and/or acid.

A third step BS3b of the process includes treating and rinsing critical regions with an aqueous solution of chlorine dioxide having a chlorine dioxide concentration in the range between 0.2 ppm and 0.4 ppm for a treatment time that is preferably five to ten minutes.

A fourth step BS4b of the process includes a final rinsing of the system components 6.

In the aforesaid steps, the treatment medium is guided through the system components 6 in a sealed flow path. This procedure is typically called “CIP cleaning.”

The process of FIG. 3 also offers significant benefits in terms of saving energy and cost because the currently normal treatment of the system components with hot water and/or steam as the disinfectant lasting twenty to thirty minutes can be dispensed with.

In all the methods described herein, chlorine dioxide concentration is no greater than 0.8 ppm. This is well below the 1.5 ppm concentration at which the disadvantages of chlorine dioxide noticeably assert themselves. These disadvantages include corrosion of steel components and unpleasant odors. Despite this, a surprisingly high treatment quality is achieved while reducing the corrosion and unpleasant odors associated with chlorine dioxide use at higher concentrations.

A particular advantage of the methods described herein is that the temperature of the treatment medium has no substantial impact on the quality of the treatment. Devices for controlling or heating the treatment medium are therefore no longer needed and can be dispensed with. A further advantage is that no sterile water is required for the treatment medium. Sterilizing water is expensive. Thus, avoiding the need for copious quantities of sterile water allows reaping of significant savings in both energy consumption and financial cost.

Only a limited number of exemplary embodiments have been described herein. The invention is not circumscribed by the described embodiments, but rather by the following claims.

Having described the invention, and a preferred embodiment thereof, what is claimed as new, and secured by Letters Patent, is:

1-9. (canceled)

10. A method for processing a structure, said method comprising treating said structure, wherein treating said structure comprises at least one of cleaning said structure, disinfecting said structure, and sterilizing said structure, wherein said structure is selected from the group consisting of a food package and a critical region of a system component of a container treatment system, wherein treating said structure comprises selecting an aqueous solution of chlorine dioxide having a chlorine dioxide concentration that is between 0.4 ppm-0.8 ppm as a treatment medium, and, in at least one treatment step, treating said structure with said treatment medium.

11. The method of claim 10, wherein treating said structure with said treatment medium comprises using said treatment medium in at least a first treatment step and a second treatment step, wherein in said first treatment step, said treatment medium has a chlorine dioxide concentration between 0.4 ppm and 0.8 ppm, wherein in said second treatment step, said treatment medium has a chlorine dioxide concentration between 0.2 ppm and 0.4 ppm, and wherein said second treatment step is subsequent to said first treatment step.

12. The method of claim 11, further comprising causing said first treatment step to last between one and two seconds, and causing said second treatment step to last half a second.

13. The method of claim 11, further comprising causing said first treatment step to last between one and two seconds, and causing said second treatment step to last between one and two seconds.

14. The method of claim 10, wherein treating said structure with said treatment medium comprises using said treatment medium in at least a first treatment step, wherein said first treatment step has a treatment time between one and two seconds.

15. The method of claim 10, wherein treating said structure with said treatment medium comprises causing said at least one treatment step to last between five minutes and ten minutes.

16. The method of claim 10, further comprising selecting said structure to be a food package, wherein said food package is a container, wherein said container is one of an unsoiled container and a cleaned container, wherein a first treatment step comprises said step of treating said structure with said treatment medium, wherein said method further comprises a second treatment step and a third treatment step, wherein said second treatment step follows said first treatment step, wherein said third treatment step follows said second treatment step, wherein said second treatment step comprises a treatment step that uses an aqueous solution of chlorine dioxide, and wherein said third treatment step comprises removing said aqueous solution of chlorine dioxide.

17. The method of claim 16, wherein removing said aqueous solution of chlorine dioxide comprises allowing said aqueous solution to drip off said container.

18. The method of claim 16, wherein removing said aqueous solution of chlorine dioxide comprises rinsing off said container.

19. The method of 18, wherein rinsing off said container comprises rinsing off with a sterile liquid.

20. The method of 18, wherein rinsing off said container comprises rinsing off with a gaseous medium.

21. The method of claim 10, further comprising selecting said structure to be at least one of a keg and a keg fitting.

22. The method of claim 21, further comprising, during treatment of said structure, draining said keg fitting to remove product residues, in a first subsequent treatment step causing said keg fitting to undergo an internal cleaning, and in a second subsequent treatment step, causing said structure to be at least one of cleaned, disinfected, and sterilized with aqueous solution of chlorine dioxide, wherein causing said keg to undergo internal cleaning comprises at least one of treating said keg with a base, treating said keg with an acid, purging said keg with a base, purging said keg with an acid, flushing said keg with water, and flushing said keg with fresh water.

23. The method of claim 21, further comprising releasing a ring seal from a location between said keg fitting and an inner surface of said keg, thereby exposing an additional surface for treatment, and treating said additional surface with said treatment medium.

24. The method of claim 10, further comprising selecting said structure to comprise system components of a filling machine, said method comprising treating critical regions of said system components in a first treatment step, wherein said first treatment step comprises treatment with one of an acid and a base, and treating said critical regions in a second treatment step, wherein said second treatment step comprises treatment with said aqueous solution of chlorine dioxide.

25. The method of claim 24, further comprising selecting said aqueous solution to have a chlorine dioxide concentration of between 0.2-0.4 ppm.

26. The method of claim 24, further comprising selecting at least one of said treatment steps to have a treatment duration of between five and ten minutes.
